(19) United States
(12) Patent Application Publication
(10) Pub. No.: US 2006/0182661 A1
(43) Pub. Date: Aug. 17, 2006

(54) Blood alcohol content (BAC) level actuated lock box

(76) Inventor: Albert Bianco Aquila, Williston Park, NY (US)

Correspondence Address: KEUSEY, TUTUNJIAN & BITETTO, P.C., 20 CROSSWAYS PARK NORTH, SUITE 210, WOODBURY, NY, 11797, US

(21) Appl. No.: 11/056,061

(22) Filed: Feb. 11, 2005

(51) Int. Cl. G01N 33/00

(52) U.S. Cl. 422/84

(57) ABSTRACT

There is provided a blood alcohol content (BAC) level actuated lock box. The BAC level actuated lock box includes a housing, a breathalyzer, and a locking device. The housing has a lockable opening for allowing an item to pass there through. The housing is for securing the item therein when the lockable opening is in a locked position. The breathalyzer is for receiving a breath sample and determining a BAC level for the breath sample. The locking device is for maintaining the lockable opening in the locked position when the determined BAC level is greater than a pre-specified BAC threshold and for unlocking the lockable opening when the determined BAC is not greater than the pre-specified BAC threshold.

### FIELD OF THE INVENTION

The present invention generally relates to lock boxes and, more particularly, to a lock box that operates using blood alcohol content (BAC) level.

### BACKGROUND OF THE INVENTION

Driving under the influence of alcohol is a significant problem with many undesirable ramifications. Most significantly, many lives are lost each year in motor vehicle accidents caused operators under the influence of alcohol. Moreover, such accidents often result in property damage to the involved motor vehicle(s) and also to other property. Additionally, insurance and other costs relating to loss of life, injuries, property damage, and litigation are also undesirable ramifications.

To prosecute a drunk driver or a driver who is impaired due to alcohol, states have enacted various laws that generally require that a motor vehicle operator have a blood alcohol content (BAC) level above a specified limit. However, despite enforcement of such laws and prosecution of corresponding violators, driving under the influence of alcohol still remains a significant problem plaguing society. Moreover, in cases where a drunk driver is apprehended by a law enforcement agency, typically such apprehension occurs when the driver is already driving and posing a great danger to the public at large.

Accordingly, it would be desirable and highly advantageous to have an apparatus and method for preventing someone under the influence of alcohol from operating a motor vehicle. Preferably, the apparatus and method would prevent a person under the influence of alcohol from even being able to start a motor vehicle in the first place. Moreover, it would be preferable if the apparatus and method could be used to prevent individuals from accessing certain items if their BAC level is above a pre-specified (e.g., legal) threshold.

### SUMMARY OF THE INVENTION

These and other drawbacks and disadvantages of the prior art are addressed by the present invention, which is directed to a blood alcohol content (BAC) level actuated lock box.

According to an aspect of the present invention, there is provided a blood alcohol content (BAC) level actuated lock box. The BAC level actuated lock box includes a housing, a breathalyzer, and a locking device. The housing has a lockable opening for allowing an item to pass there through. The housing is for securing the item therein when the lockable opening is in a locked position. The breathalyzer is for receiving a breath sample and determining a BAC level for the breath sample. The locking device is for maintaining the lockable opening in the locked position when the determined BAC level is greater than a pre-specified BAC threshold and for unlocking the lockable opening when the determined BAC is not greater than the pre-specified BAC threshold.

According to another aspect of the present invention, there is provided a method for securing an item based on blood alcohol content (BAC) level. A housing is provided having a lockable opening for allowing the item to pass there through. The housing is for securing the item therein when the lockable opening is in a locked position. A breath sample is received, and a BAC level is determined for the breath sample. The lockable opening is maintained in the locked position to secure the item therein when the determined BAC level is greater than a pre-specified BAC threshold. The lockable opening is unlocked when the determined BAC is not greater than the pre-specified BAC threshold.

These and other aspects, features and advantages of the present invention will become apparent from the following detailed description of exemplary embodiments, which is to be read in connection with the accompanying drawings.

### BRIEF DESCRIPTION OF THE DRAWINGS

The present invention may be better understood in accordance with the following exemplary figures, in which:

FIG. 1 is a diagram illustrating an exterior of a blood alcohol content (BAC) level actuated ignition key lock box 100, according to an illustrative embodiment of the present invention;

FIG. 2 is a diagram illustrating an interior of the BAC level actuated ignition key lock box 100 of FIG. 1, according to an illustrative embodiment of the present invention;

FIG. 3 is a flow diagram illustrating a blood alcohol content (BAC) level based method for securing an ignition key for a motor vehicle, according to an illustrative embodiment of the present invention; and

FIG. 4 is a flow diagram illustrating an identity verification feature for the lock box 100 of FIG. 1, according to an illustrative embodiment of the present invention.

### DETAILED DESCRIPTION

The present invention is directed to a blood alcohol content (BAC) level actuated lock box. In preferred embodiments of the present invention described herein, the lock box is used to secure an ignition key therein. However, it is to be appreciated that the present invention is not limited solely to securing ignition keys and, thus, other items may also be secured in the lock box according to the present invention while maintaining the scope thereof. For example, law enforcement officers may be required to provide an acceptable BAC limit at the beginning of their shift in order to retrieve a firearm locked in a lock box according to the present invention. These and other applications for the lock box according to the present invention are described in further detail herein. Thus, it is to be appreciated that given the teachings of the present invention provided herein, one of ordinary skill in the related art will contemplate these and various other application for a lock box according to the present invention, while maintaining the scope of the present invention.

With respect to the preferred embodiment directed to ignition keys, the present invention advantageously aids in the prevention of alcohol-induced drunk driving and driving under the influence of alcohol by restricting a motor vehicle operator from accessing the ignition key of the motor vehicle. In this way, a person who is drunk or otherwise under the influence of alcohol may be prevented from operating a motor vehicle in the first place. As drunk drivers are often caught by law enforcement only after they are behind the wheel and in many instances only after they have been involved in an accident, the present invention advantageously reduces the risk to the public by preventing the driver from accessing the ignition key.

According to one illustrative embodiment of the present invention, prior to consuming alcoholic beverages, the operator of the motor vehicle places the ignition key for the motor vehicle into the box, which then locks. The box unlocks, granting access to the ignition key, only if breath analysis, performed by an onboard breathalyzer, results in a blood alcohol content (BAC) value that is less than a pre-specified limit. The pre-specified limit may be set to the legal limit for driving under the influence of alcohol in the particular state in which the present invention is being utilized or to some other value, e.g., to a lower value in the case of businesses that regularly utilize motor vehicles and employ the present invention to ensure that their employees who operate such vehicles are not impaired to a degree even more stringent than corresponding legal limits. The pre-specified limit may be changed when a user of the present invention intends to utilize the present invention in a different state, and so forth.

The lock box according to the present invention may be of any dimensions and any shape. In one illustrative embodiment of the present invention, the present invention is implemented as a small lock box, with dimensions and shape similar to a wallet. However, as noted herein, the present invention is not limited to the preceding dimensions and shapes.

The present description illustrates the principles of the present invention. It will thus be appreciated that those skilled in the art will be able to devise various arrangements that, although not explicitly described or shown herein, embody the principles of the invention and are included within its spirit and scope.

All examples and conditional language recited herein are intended for pedagogical purposes to aid the reader in understanding the principles of the invention and the concepts contributed by the inventor to furthering the art, and are to be construed as being without limitation to such specifically recited examples and conditions.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that the block diagrams presented herein represent conceptual views of illustrative circuitry embodying the principles of the invention. Similarly, it will be appreciated that any flow charts, flow diagrams, state transition diagrams, pseudocode, and the like represent various processes which may be substantially represented in computer readable media and so executed by a computer or processor, whether or not such computer or processor is explicitly shown.

The functions of the various elements shown in the figures may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (“DSP”) hardware, read-only memory (“ROM”) for storing software, random access memory (“RAM”), and non-volatile storage.

Other hardware, conventional and/or custom, may also be included. Similarly, any switches shown in the figures are conceptual only. Their function may be carried out through the operation of program logic, through dedicated logic, through the interaction of program control and dedicated logic, or even manually, the particular technique being selectable by the implementer as more specifically understood from the context.

In the claims hereof, any element expressed as a means for performing a specified function is intended to encompass any way of performing that function including, for example, a) a combination of circuit elements that performs that function or b) software in any form, including, therefore, firmware, microcode or the like, combined with appropriate circuitry for executing that software to perform the function. The invention as defined by such claims resides in the fact that the functionalities provided by the various recited means are combined and brought together in the manner which the claims call for. It is thus regarded that any means that can provide those functionalities are equivalent to those shown herein.

In the illustrative embodiments described herein, the blood alcohol content (BAC) level actuated ignition key lock box has rectangular dimensions, and is similar in size to a wallet. However, it is to be appreciated that the present invention is not limited solely to the shapes, dimensions, and other particulars shown and described herein and, thus, other shapes, dimensions, and so forth may also be employed in accordance with the present invention while maintaining the scope of the present invention. Moreover, it is to be further appreciated that the present invention may be used to lock other items, in addition to, or in place of, an ignition key. That is, the present invention is not limited to securing ignition keys therein and, thus, other items may also be secured therein while maintaining the scope of the present invention.

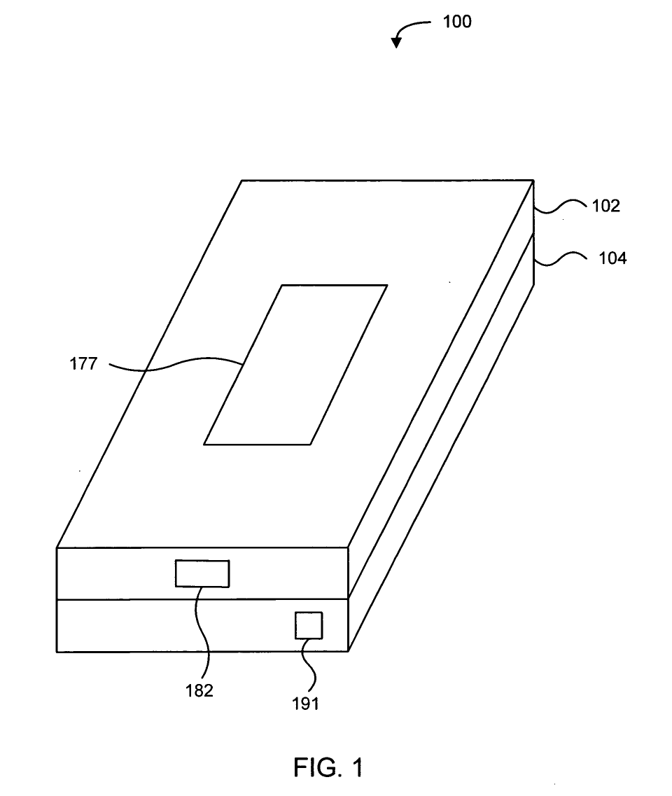

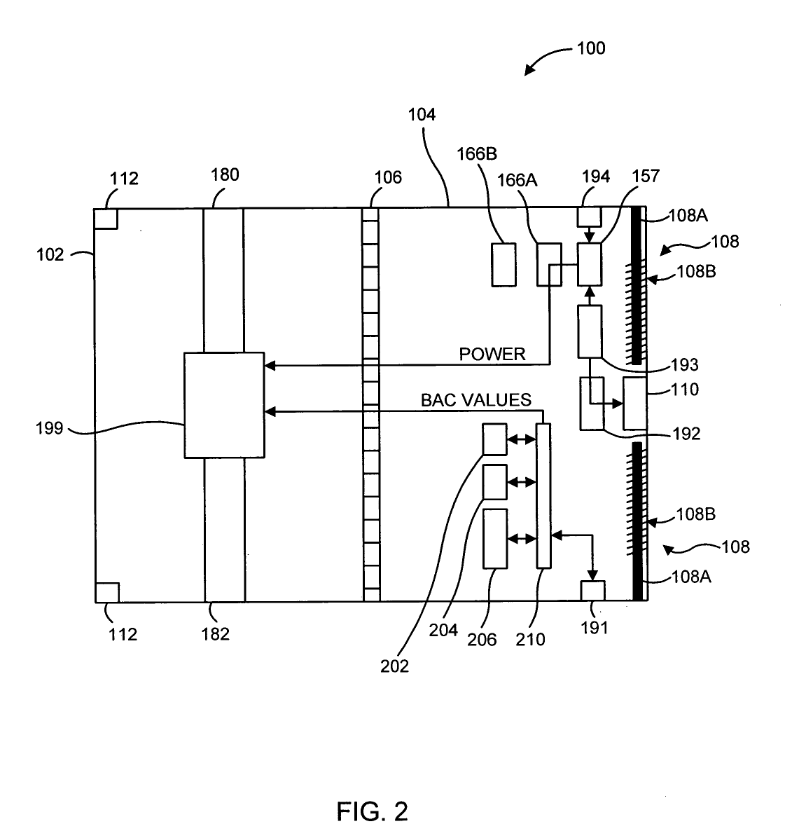

FIG. 1 is a diagram illustrating an exterior of a blood alcohol content (BAC) level actuated ignition key lock box 100, according to an illustrative embodiment of the present invention. FIG. 2 is a diagram illustrating an interior of the BAC level actuated ignition key lock box 100 of FIG. 1, according to an illustrative embodiment of the present invention. It is to be appreciated that the perspective view of the lock box 100 presented in FIG. 1 represents the lock box 100 in a closed and locked condition. In contrast, the view of the lock box depicted in FIG. 2 represents the lock box 100 in an open and unlocked condition.

As shown in FIG. 1 and/or FIG. 2, the lock box 100 includes an upper half housing portion 102, a lower half housing portion 104, and a hinge 106 for connecting the upper half housing portion 102 to the lower half housing portion 104. It is to be appreciated that the hinge 106 is disposed internal to the lock box 100 to prevent tampering there with. However, it is to be further appreciated that the present invention is not limited to internal disposed hinges and, thus, other fastening means including, but not limited to, external hinges and so forth may also be employed in accordance with the present invention while maintaining the scope of the present invention. The upper half housing portion 102 includes a display device 177, where the BAC value is displayed for an operator that has submitted a breath sample.

The lock box 100 further includes an air intake tunnel 180, an air exhaust tunnel 182, a breathalyzer 199, an electromagnet solenoid device (hereinafter “solenoid”) 110, a switching device 192, a battery 193, a power management device 157, at least one input/output data port (hereinafter “input/output data port”) 191, at least one power port (hereinafter “power port”) 194, an identity verification apparatus 166, and two spring-loaded pins 108. Each of the two spring-loaded pins 108 includes a pin 108A and a spring 108B.

Moreover, in a GPS enabled embodiment of the present invention, the lock box 100 further includes a Global Positioning System (GPS) chip/device or other location determining chip/device (hereinafter “GPS device”) 202 and at least one memory device (hereinafter “memory”) 204, and at least one processor (hereinafter “processor”) 206, all interconnected to the breathalyzer 199 via a bus 210. It is to be appreciated that the GPS enabled embodiment is an optional aspect of the lock box 100 of the present invention. The GPS enabled embodiment is further described herein below.

The two spring-loaded pins 108 are located in respective corners of the lower half housing portion 104 on the side opposing the hinge 106. The pins 108A lock the lower half housing portion 104 to the upper half housing portion 102 when in an extended position. The pins 108A are biased by the springs 108B to normally be in the extended position. That is, the spring force of the springs 108B act to keep the pins 108A in the extended position, until the pins 108A are retracted as described herein below. The pins 108A (as well as the springs 108B) run parallel to the hinge 106 and retract towards the center of the side on which the pins 108A are located. The pins 108A are retracted by the solenoid 110. The solenoid 110 is located in between the pins 108A on the lower half housing portion 104.

The pins 108A, when retracted, allow the upper half housing portion 102 to close, making full contact with the lower half housing portion 104. When the pins 108A are not retracted by the solenoid 110, the springs 108B push the pins 108A into pin-receiving apertures 112 that are located in respective corners of the upper half housing portion 102.

When the pins 108A are located inside the pin-receiving apertures 112 of the upper half housing portion 102 of the lock box 100, the lock box 100 is locked and not capable of being opened. When the pins 108A are retracted, the pins 108A recede from the pin-receiving apertures 112, allowing the two halves 102 and 104 to pivot along the hinge 106 and separate.

A description will now be given regarding the breathalyzer 199 and the display device 177, according to an illustrative embodiment of the present invention.

The breathalyzer 199 is located in the center of the upper half housing portion 102. The air intake tunnel 180 is located in a middle area of the upper half housing portion 102 and runs parallel to the hinge 106. The air exhaust tunnel 182 is located in the middle of the upper half housing portion 102, parallel to the hinge 106, and opposite to the air intake tunnel 180. The air exhaust tunnel 182 is a continuation of the air intake tunnel 180, interrupted only by the centrally located breathalyzer 199. In some embodiments of the present invention, the air intake tunnel 180 is adapted for use with personal tubes/straws (hereinafter “personal tubes”) so that germs are not spread in the event that the lock box is to be used by more than one person (e.g., fleet applications as described herein).

A sample breath is submitted to the breathalyzer 199 for analysis via the air intake tunnel 180. The breathalyzer 199 analyzes the sample breath and outputs a switch value (described in further detail herein below) to the solenoid 110 and also outputs a measured/calculated BAC value (hereinafter “BAC value”) corresponding to the sample breath to the display device 177. Exhaust air is released through the air exhaust tunnel 182.

The display device 177 is located on the outer side of the upper half housing portion 102, flush to the surface of the upper half housing portion 102. The BAC value corresponding to a submitted breath sample is displayed on the display device 177. It is to be appreciated that the display device 177 is optional, and is not necessary for the operation of the lock box 100. That is, irrespective of whether a user is made aware of their measured BAC level via the display device 177, the lock box 100 will remain closed until a sample breath is submitted that meets a pre-specified BAC threshold, as further described in detail herein below. It is to be appreciated that the display device 177 is not limited to any particular technology or type of display device, and may include, for example, a Liquid Crystal Display (LCD), a plasma screen, and so forth

The breathalyzer 199 is preferably battery operated, e.g., by the battery 193. However, other means to power the breathalyzer 199 may also be employed while maintaining the scope of the present invention. For example, in some embodiments of the present invention, the lock box 100 may be permanently installed in a motor vehicle, in which case the lock box 100 may be hard-wired to a power source within the motor vehicle. In other embodiments of the present invention, power to the lock box 100 may be supplied via 110 VAC power or 12 VDC power (the latter, for example, from a motor vehicle battery) or some other voltage. In such a case, the lock box 100 receives power via power port 194. It is to be appreciated that battery 193 may include more than one battery. In the illustrative embodiment of FIG. 2, the battery 193 is located in the lower half housing portion 104.

Thus, it is to be appreciated that the power port 194 may also be configured to receive AC power. In such a case, the power management device 157 determines the power source to be used. If the power port 194 is unconnected to any external source, then power is provided to the lock box 100 and elements thereof by the battery 193. Otherwise, if the power port 194 is connected to an external source, the power management device detects this condition, evaluates the voltage supplied (AC or DC, and the corresponding voltage level) and performs the necessary power conditioning operations including, but not limited to, rectifying, converting from AC to DC, stepping-up operations, stepping-down operations, and so forth. In the event of a lack of power supplied to the lock box 100, either because, e.g., the battery is dead or external power is unavailable, then an item such as an ignition key locked therein will remain locked therein.

A description will now be given regarding the input/output data port 191, according to an illustrative embodiment of the present invention.

The input/output data port 191 is located on the short side of the lower half housing portion 104. The input/output data port 191 may be any type of data port including, but not limited to, a Universal Serial Bus, a port compliant with IEEE1394 (e.g., FIREWIRE), and so forth.

The input/output data port 191 may be used to input data to the lock box 100 and to output data from the lock box 100.

For example, the input/output data port 191 may be used to connect the lock box 100 to a computer (not shown) to allow updated information regarding various legal BAC limits for any state of the United States to be input to the lock box 100. This allows the operator to update the lock box 100 so as to stay current with the laws of his/her state and also allows the operator to change the pre-specified BAC threshold level if the operator is traveling to a state with different legal BAC standards than the operator's home state. The BAC limit information may be provided, e.g., by the manufacturer through their website or via a website(s) maintained by or otherwise involved with a corresponding governmental entity. Moreover, a manufacturer, state, or some other entity may maintain a secure website that uses a secure protocol to interface with a lock box 100 to set/change the BAC level to prevent unauthorized setting/changing (hereinafter also collectively referred to as “changing”) of the pre-specified BAC threshold level. Additionally, the changing of the pre-specified BAC threshold level may require that the lock box 100 be sent back to the manufacturer or be brought to certain designated service locations to prevent unauthorized changing of the pre-specified BAC threshold level. Thus, it is preferable but not mandatory that the BAC limit information is provided to the lock box 100 in a manner that prevents false (e.g., too low or none at all) BAC limits from being input to the lock box 100. For example, passwords, encryption and so forth may be used to set and/or update the BAC information used by the breathalyzer.

Moreover, in some illustrative embodiments of the present invention, the lock box 100 may further include a memory device (such as, e.g., memory 204 or some other memory device) for storing previously measured BAC levels, and the input/output data port 191 may be used to output these previously measured BAC levels. Such data may be admissible in court or may have some other use, such as monitoring employees when the present invention is used in a company-wide application as described in further detail herein below.

A description will now be given regarding BAC levels used by the present invention, according to an illustrative embodiment of the present invention.

As noted above, the BAC level(s) employed by the present invention may optionally be changed using the input/output data port 191.

A factory-default BAC threshold may be set at the factory. The factory-default BAC threshold may be based on, e.g., the national average for BAC limits, and so forth.

It is preferable but not mandatory, that the lock box 100 includes a factory-default lowest BAC threshold limit that is always in place (i.e., that cannot be set lower, but can be set higher if necessary or desired). The factory-default lowest BAC threshold limit may coincide to, for example, the lowest known BAC limit from among all of the states in the United States. In this way, some BAC level (i.e., the factory-default lowest BAC threshold limit) will always be in place to allow the lock box to function as intended.

It is to be appreciated that BAC levels corresponding to other locales (e.g., other countries, etc.) may also be employed, while maintaining the scope of the present invention. Thus, in addition to other states, the present invention may also be used in other countries.

A description will now be given of a GPS enabled embodiment of the present invention. In the GPS enabled embodiment, the lock box 100 further includes the GPS device 302, the memory 304, and the processor 306. The legal BAC limits for each state in the United States and/or for other locales/countries are stored in the memory 304. A signal indicative of a current location is output by the GPS device 302 and used by the processor 306 to select the corresponding applicable legal BAC limit from the memory 304. This may be performed using a mapping or any other correlation functions and means, as readily determined by one of ordinary skill in the art while maintaining the scope of the present invention. The corresponding applicable legal BAC limit is then provided to the breathalyzer 199 for subsequent use when analyzing sample breaths.

A description will now be given regarding the operation of the breathalyzer 199, the switching device 192, the solenoid 110, and the battery 193, according to an illustrative embodiment of the present invention.

In addition to analyzing a breath sample from a potential motor vehicle operator and providing a corresponding measured BAC value to the display device 177 for displaying thereon, the breathalyzer 199 is capable of producing an electronic signal that has values of “0” or “1” (or any other values), depending on a result of the analysis (the resultant BAC value). That is, if the driver's BAC value is less than the driver's state's legal BAC limit for driving under the influence of alcohol, then the value of the signal produced will be “0”. In contrast, if the driver's BAC value is equal to or greater than the driver's state's legal BAC limit for driving under the influence of alcohol, the value of the signal produced will be “1”. The “0” and “1” values output from the breathalyzer 199 are also referred to herein as “switch values”.

The switching device 192 is included in the upper half housing portion 102. The switching device 192 receives the electronic “0” or “1” signal produced by the breathalyzer 199, and either: (a) upon receipt of a “0” signal, allows an electrical current to flow from the battery 193 to the solenoid 110 which retracts the pins 108A to allow the lock box 100 to be opened; or (b) upon receipt of a “1” signal, does not allow any current to flow from the battery 193 to the solenoid 110, thus leaving the pins 108A in the extended (i.e., locked) position (due to the spring force exerted by springs 108B) to cause the lock box 100 to remained closed.

When the pins 108A are retracted, an internal timing device located in the switching device 192 keeps the pins 108A retracted for a total of 5 seconds (or other pre-specified time period), after which, the switching device 192 no longer allows the flow of electrical current to the solenoid 110, allowing the spring driven pins 108A to return to their “locked” position (by the spring force exerted by springs 108B).

It is to be appreciated that the switching device 192 may be implemented using logic circuitry (e.g., if-then logic circuitry, etc.), Application Specific Integrated Circuits (ASICs), comparators, and so forth.

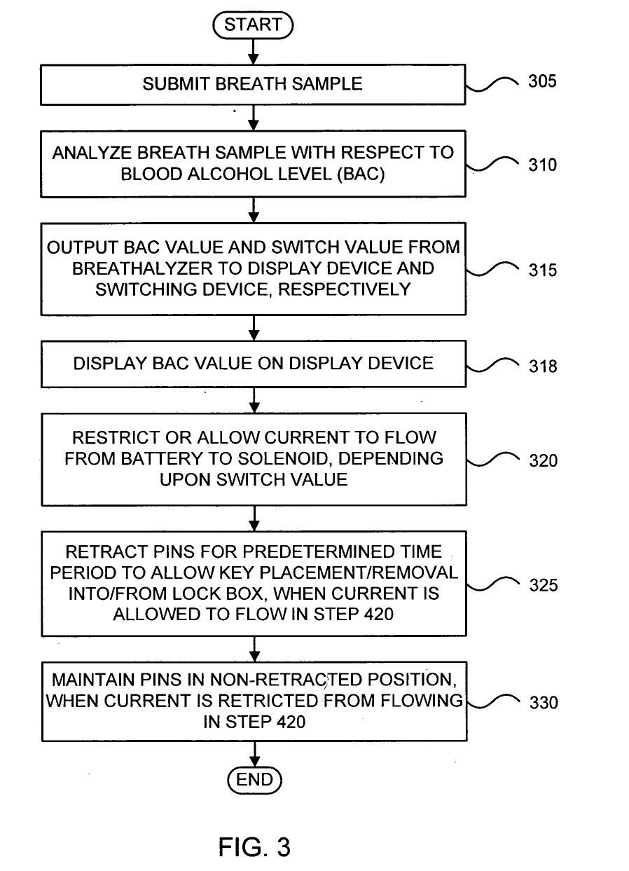

FIG. 3 is a flow diagram illustrating a blood alcohol content (BAC) based method for securing an ignition key for a motor vehicle, according to an illustrative embodiment of the present invention.

A breath sample is submitted to an ignition key lock box 100, by a user (potential motor vehicle operator) blowing air into the air intake tunnel 180 (step 305). The breath sample is analyzed by the breathalyzer 199 (step 310). A BAC value and a switch value (“0” or “1”) are respectively output from the breathalyzer 199 to the display device 177 and the switching device 192, as outputs of the analysis performed at step 310 (step 315). The BAC value is displayed on the display device 177 (step 318). The switching device 192 either restricts or allows current to flow from the battery 193 to the solenoid 110, depending upon the switch value (step 320). If current is allowed to flow from the battery 193 to the solenoid 110 at step 320, then the pins 108A are retracted for a predetermined time period to allow an ignition key for a motor vehicle to be placed within the lock box 100 or removed from the lock box 100 (step 325). Otherwise, if current is restricted from flowing from the battery 193 to the solenoid 110 at step 320, then the pins 108A are maintained in the non-retracted (i.e., extended) position (step 330).

With respect to step 325, once the ignition key is placed into the lock box 100, the user must hold the lock box 100 closed until the pins 108 return to the non-retracted (i.e., locked) position.

In the case when step 325 is performed for the purpose of initially placing the ignition key in the lock box 100, then steps 305 through 330 are repeated to obtain the ignition key from the lock box 100 when the user intends to operate the corresponding motor vehicle.

A description will now be given regarding the identify verification apparatus 166. In some embodiments of the present invention, the identity verification apparatus 166 may be employed. The identity verification apparatus 166 is utilized to ensure, e.g., that the person who is attempting to unlock the lock box 100 to retrieve an item (e.g., ignition key) locked therein is the same person who locked the item in the lock box 100 in the first place. Accordingly, a technology or approach that is not easily circumvented is preferably employed. For example, in a preferred embodiment of the present invention, biometrics are employed to verify identity. The use of biometrics avoids easy circumvention of the identity verification, as compared to other security indicia such as, e.g., passwords. Fingerprints are one example of a biometric that may be used to verify identity. Of course, the present invention is not limited to the preceding biometrics and, thus, other biometrics may also be employed, as well as non-biometrics, to verify identity in accordance with the present invention, while maintaining the scope of the present invention.

The identity verification apparatus 166 comprises a switching device 166A, and a biometric verification device (e.g., a fingerprint reader) 166B. The identify verification apparatus 166 is intended to cooperate with the battery 193 and the breathalyzer 199 as described herein.

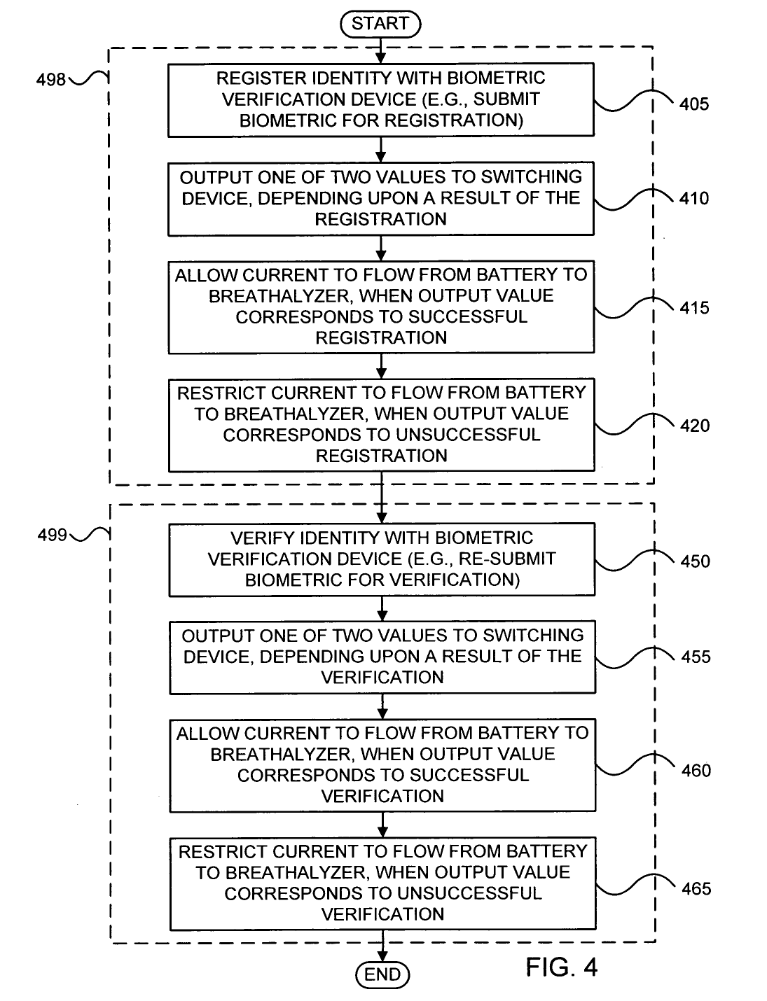

FIG. 4 is a flow diagram illustrating an identity verification feature for the lock box 100 of FIG. 1, according to an illustrative embodiment of the present invention.

To lock an ignition key (or other item) in the lock box 100 when the identify verification apparatus 166 is being utilized, a user must first register his or her identify with the biometric verification device 166B in a registration stage 498 (step 405). Step 405 may involve, e.g., the user submitting one or more fingerprints to the device 166B. Upon the registration of one or more fingerprints, the biometric verification device 166B outputs one of two possible output values in an output signal provided to the switching device 166A (step 410). In this example, the output value from the biometric verification device 166B upon a successful registration is a “1”. Otherwise, it would be a “0” for an unsuccessful registration. If the switching device 166A receives an output signal having a value of “1”, then the switching device 166A allows an electrical current to flow from the battery 193 to the breathalyzer 199 to power the breathalyzer 199 (step 415). If the switching device 166A receives an output signal having a value of “0”, then the switching device 166A restricts the electrical current from flowing from the battery to the breathalyzer 199 (step 420). Once the breathalyzer 199 is powered, the user may utilize the lock box 100 as otherwise described herein to lock the ignition key therein.

To retrieve an ignition key (or other item) that is already locked in the lock box 100 when the identify verification apparatus 166 is being utilized, the user submits one or more fingerprints to the biometric verification device 166B in a verification stage 499 (step 450). Upon the verification of the one or more fingerprints, the biometric verification device 166B outputs one of two possible output values in an output signal provided to the switching device 166A (step 455). If the user is successfully verified by the identify verification apparatus 166 in the verification stage, then the identify verification apparatus 166 outputs a signal having a value of “1”. Otherwise, if the user is not successfully verified by the identity verification apparatus 166, then the identify verification apparatus 166 outputs a signal having a value of “0”. If the switching device 166A receives an output signal having a value of “1”, then the switching device 166A allows an electrical current to flow from the battery 193 to the breathalyzer 199 to power the breathalyzer 199 (step 460). If the switching device 166A receives an output signal having a value of “0”, then the switching device 166A restricts the electrical current from flowing from the battery to the breathalyzer 199 (step 465). Once the breathalyzer 199 is powered, the user may utilize the lock box 100 as otherwise described herein to retrieve the ignition key.

The registration stage may be implemented using software on a secure website (e.g., the manufacturer's website). This ensures that a designated (i.e., currently registered) user having their ignition key locked therein is not replaced in “mid-use” by another person. Another approach to avoid the preceding is to have the registration software on the device 166B itself, but configured so that registration cannot be changed in “mid-use”. In such a case, the registered user may only be changed after a pre-specified number of hours (with the intent that the operator may now be sober after such time), and/or upon accessing a secure website or bringing the lock box 100 to an authorized service center to change the registered user.

A description will now be given regarding various other aspects, implementations, and applications of the present invention.

In some embodiments of the present invention, alternatively to the element layout shown and described herein, the breathalyzer may separate and apart from the lock box and may connect to the box via the input/output data port 191 or some other means.

Thus, a lock box according to the present invention can exist in a variety of shapes and sizes, with either an internal or external breathalyzer, so that items larger than automobile keys may be locked and accessed only when the operator's BAC value is lower than a specified limit. For example, firearms may be locked therein at the start of a shift for a law enforcement officer and only obtained by the officer of the officer is capable of providing an acceptable BAC level.

Of course, it is to be appreciated that the locations of the elements of the lock box are not critical as long as the functions of the elements are not impaired. Thus, depending upon the size of the box, one or more of the components originally described as being in the upper half of the box may be located in the lower half while maintaining the scope of the present invention. It is to be appreciated that instead of the lock box opening by two halves (e.g., upper and lower halves as described herein) separating, a lockable opening may be utilized. The lockable opening may be sized to correspond to any size depending on the application. Moreover, the actual opening of the lock box by separating the upper and lower halves as described herein may be considered to employ a lockable opening.

It is to be further appreciated that while the present invention is described herein with respect to the solenoid 110, the switching device 192, the battery 193, the spring-loaded pins 108, and the pin-receiving apertures 112 for facilitating the locking and unlocking of the lock box 100, other locking means and devices as readily contemplated by those of ordinary skill in the art may also be employed while maintaining the scope of the present invention.

Another variation to be appreciated is that the pins 108A may be replaced with cylindrical or other-shaped permanent magnets so that the magnets can be retracted or extended within a solenoid “tube” by simply reversing the electrical field. In such case, the “switch values” are used to reverse the magnetic field applied to the permanent magnet so that the permanent magnet retracts and extends as desired. These and other variations are readily contemplated by those of ordinary skill in the related art and may be readily employed by the present invention while maintaining the scope thereof.

It is to be still further appreciated that the present invention is not limited to individual use as described herein. That is, the present invention may be utilized by companies (e.g., trucking company, taxicab companies, etc.), organizations (police department, fire department, etc.), and so forth, that would like to ensure that motor vehicle operators associated there with are not operating motor vehicles while drunk or otherwise impaired by alcohol. In such applications, the lock box according to the present invention may be utilized, for example, at shift changes where the person “coming off” of a shift locks the key in the lock box so that a person “coming on” a shift then has to unlock the lock box by providing an acceptable BAC level. Given the teachings of the present invention provided herein, these and other implementations of the present invention are readily determined by those of ordinary skill in the related art, while maintaining the scope of the present invention.

These and other features and advantages of the present invention may be readily ascertained by one of ordinary skill in the pertinent art based on the teachings herein. It is to be understood that the teachings of the present invention may be implemented in various forms of hardware, software, firmware, special purpose processors, or combinations thereof.

Most preferably, the teachings of the present invention are implemented as a combination of hardware and software. Moreover, the software is preferably implemented as an application program tangibly embodied on a program storage unit. The application program may be uploaded to, and executed by, a machine comprising any suitable architecture. Preferably, the machine is implemented on a computer platform having hardware such as one or more central processing units (“CPU”), a random access memory (“RAM”), and input/output (“I/O”) interfaces. The computer platform may also include an operating system and microinstruction code. The various processes and functions described herein may be either part of the microinstruction code or part of the application program, or any combination thereof, which may be executed by a CPU. In addition, various other peripheral units may be connected to the computer platform such as an additional data storage unit and a printing unit.

It is to be further understood that, because some of the constituent system components and methods depicted in the accompanying drawings are preferably implemented in software, the actual connections between the system components or the process function blocks may differ depending upon the manner in which the present invention is programmed. Given the teachings herein, one of ordinary skill in the pertinent art will be able to contemplate these and similar implementations or configurations of the present invention.

Although the illustrative embodiments have been described herein with reference to the accompanying drawings, it is to be understood that the present invention is not limited to those precise embodiments, and that various changes and modifications may be effected therein by one of ordinary skill in the pertinent art without departing from the scope or spirit of the present invention. All such changes and modifications are intended to be included within the scope of the present invention as set forth in the appended claims.

What is claimed is:

1. A blood alcohol content (BAC) level actuated lock box, comprising:
a housing having a lockable opening for allowing an item to pass there through, the housing for securing the item therein when the lockable opening is in a locked position;
a breathalyzer for receiving a breath sample and determining a BAC level for the breath sample; and
a locking device for maintaining the lockable opening in the locked position when the determined BAC level is greater than a pre-specified BAC threshold and for unlocking the lockable opening when the determined BAC is not greater than the pre-specified BAC threshold.

2. The BAC level actuated lock box according to claim 1, wherein said housing further comprises a tunnel for directing the breath sample towards the breathalyzer for analysis and for directing exhaust air away from the breathalyzer.

3. The BAC level actuated lock box according to claim 3, wherein the tunnel comprises:
an air intake tunnel for receiving the breath sample from a user and directing the breath sample to the breathalyzer for the analysis; and
an air exhaust tunnel for directing the exhaust air away from the breathalyzer.

4. The BAC level actuated lock box according to claim 1, wherein the air exhaust tunnel is a continuation of the air intake tunnel that is interrupted by an intermediate position of the breathalyzer.

5. The BAC level actuated lock box according to claim 1, wherein the housing comprises:
an upper half housing portion;
a lower half housing portion; and
a hinge for connecting the upper half housing portion to the lower half housing portion,
wherein the lockable opening is formed from the upper half housing portion and the lower half housing portion.

6. The BAC level actuated lock box according to claim 5, wherein said locking device locks the upper half housing portion to the lower half housing portion.

7. The BAC level actuated lock box according to claim 5, wherein said lock comprises:
an aperture disposed on one of the upper half housing portion and the lower half housing portion; and
at least one pin, disposed on another one of the upper half housing portion and the lower half housing portion, having an extended and a retracted position for respectively locking and unlocking the lockable opening by extending into and retracting from the pin-receiving aperture.

8. The BAC level actuated lock box according to claim 7, wherein said lock further comprises at least one spring for normally biasing the at least one pin to extend into the pin-receiving aperture.

9. The BAC level actuated lock box according to claim 7, wherein said lock further comprises:
a solenoid for maintaining the at least one pin in the extended position or placing the lock in the retracted position, depending upon a control signal; and
a switching device for generating the control signal to one of restrict and allow current to flow from a power source to the solenoid device based on an output of said breathalyzer.

10. The BAC level actuated lock box according to claim 1, wherein the housing is adapted to secure any one of the ignition key and a firearm.

11. The BAC level actuated lock box according to claim 1, wherein said breathalyzer is adapted to use personal tubes.

12. The BAC level actuated lock box according to claim 1, wherein said breathalyzer comprises a factory-default BAC threshold.

13. The BAC level actuated lock box according to claim 12, wherein the factory-default BAC threshold is based on a national average for BAC limits.

14. The BAC level actuated lock box according to claim 1, wherein said breathalyzer comprises a factory-default lowest BAC threshold limit such that any subsequently used BAC thresholds by said breathalyzer are never less than the factory-default lowest BAC threshold limit.

15. The BAC level actuated lock box according to claim 1, further comprising
at least one memory device for storing a plurality of BAC legal limits, each of the plurality of BAC legal limits corresponding to at least one of a plurality of locales;
a Global Positioning System (GPS) device for determining a current position of a user of the lock box; and
at least one processor for selecting one of the plurality of BAC legal limits for use by said breathalyzer based upon the current position determined by the GPS device.

16. The BAC level actuated lock box according to claim 1, wherein the breathalyzer is disposed one of internal and external with respect to the housing.

17. The BAC level actuated lock box according to claim 1, wherein said locking device comprises:
a lock having an extending position and a retracted position for respectively locking and unlocking the lockable opening;
a solenoid for maintaining the lock in the extended position or placing the lock in the retracted position, depending upon a control signal; and
a switching device for generating the control signal to one of restrict and allow current to flow from a power source to the solenoid based on an output of said breathalyzer.

18. The BAC level actuated lock box according to claim 17, wherein said breathalyzer outputs a first value when the determined BAC level is greater than the pre-specified BAC threshold and a second value when the determined BAC level is not greater than the pre-specified BAC threshold.

19. The BAC level actuated lock box according to claim 18, wherein the first value and the second value output from said breathalyzer are binary values.

20. The BAC level actuated lock box according to claim 17, wherein said housing comprises an aperture, and the lock comprises a cylindrical permanent magnet for extending into the aperture to lock the lockable opening when the lock is in the extended position and for retracting from the aperture to unlock the lockable opening when the lock is in the retracted position.

21. The BAC level actuated lock box according to claim 1, wherein said housing comprises a pin-receiving aperture, and the lock comprises:
at least one pin for extending into the pin-receiving aperture when the lock is in an extended position and for retracting from the pin-receiving aperture to unlock the lockable opening; and
at least one spring for normally biasing the at least one pin to extend into the pin-receiving aperture.

22. The BAC level actuated lock box according to claim 1, further comprising an input/output data port.

23. The BAC level actuated lock box according to claim 22, wherein said breathalyzer is disposed external to said housing, and is electrically connected to the lock box via the input/output data port.

24. The BAC level actuated lock box according to claim 22, wherein said input/output data port is for receiving updated BAC limit information for use by said breathalyzer.

25. The BAC level actuated lock box according to claim 24, further comprising a secure communication device for allowing secure communication with at least one remote location with respect to receiving the updated BAC information.

26. The BAC level actuated lock box according to claim 22, further comprising a memory device for storing previously measured BAC levels, and wherein said input/output data port is for outputting the previously measured BAC levels.

27. The BAC level actuated lock box according to claim 1, further comprising a display device for displaying a BAC value for an operator that has submitted a breath sample.

28. The BAC level actuated lock box according to claim 27, wherein said display device comprises at least one of a Liquid Crystal Display (LCD) and a plasma screen.

29. The BAC level actuated lock box according to claim 1, further comprising an identity verification apparatus for verifying that a same individual locks the item in the lock box and unlocks the lock box to retrieve the item.

30. The BAC level actuated lock box according to claim 1, wherein said identity verification apparatus comprises:
identity verification device for verifying an identity of the individual; and
a switching device for generating the control signal to one of restrict and allow current to flow from a power source to the breathalyzer based on an output of said identity verification device, so as to render the breathalyzer unpowered and inoperable when the identity of the individual is not verified.

31. The BAC level actuated lock box according to claim 1, wherein said identity verification device comprises a fingerprint reader.

32. A method for securing an item based on blood alcohol content (BAC) level, comprising the steps of:
providing a housing having a lockable opening for allowing the item to pass there through, the housing for securing the item therein when the lockable opening is in a locked position;
receiving a breath sample and determining a BAC level for the breath sample;
maintaining the lockable opening in the locked position to secure the item therein when the determined BAC level is greater than a pre-specified BAC threshold; and
unlocking the lockable opening when the determined BAC is not greater than the pre-specified BAC threshold.

33. The method according to claim 32, further comprising the step of displaying the determined BAC level for viewing by a submitter of the breath sample.

34. The method according to claim 32, wherein the item is one of an ignition key and a firearm.

35. The method according to claim 32, wherein said method is used in a fleet application.
